(19) United States
(12) Patent Application Publication
(10) Pub. No.: US 2024/0118210 A1
(43) Pub. Date: Apr. 11, 2024

(54) FFN FLUORESCENCE RELEASE ASSAY (FFRA) AND METHODS OF USING SAME

(71) Applicants: Duke University, Durham, NC (US); The Trustees of Columbia University in the City of New York, New York, NY (US)

(72) Inventors: Gowthami Arepally, Durham, NC (US); Samuel Francis, Durham, NC (US); Christopher Hwu, Corona, NY (US); Dalibor Sames, New York, NY (US); David Sulzer, New York, NY (US)

(21) Appl. No.: 18/263,764

(22) PCT Filed: Feb. 3, 2022
(86) PCT No.: PCT/US2022/015143

§ 371 Date: Aug. 1, 2023

Related U.S. Application Data
(60) Provisional application No. 63/145,097, filed on Feb. 3, 2021.

(51) Int. Cl. G01N 21/64; C07D 221/08; G01N 33/53

(52) U.S. Cl. CPC G01N 21/6486; C07D 221/08; G01N 33/53; G01N 2400/40; G01N 2800/222

(57) ABSTRACT

Disclosed herein are methods of diagnosing a disease or condition associated with abnormal platelet activation in a subject. Also disclosed herein are methods for assessing the propensity of donor platelets to release an uptaken fluorescent false neurotransmitter (FFN).

### CROSS-REFERENCE TO RELATED APPLICATIONS

This application claims priority to U.S. Provisional Patent Application No. 63/145,097, filed Feb. 3, 2021, which is incorporated herein by reference in its entirety.

### STATEMENT REGARDING FEDERALLY SPONSORED RESEARCH

This invention was made with government support under grant MH108186 awarded by the National Institutes of Health. The government has certain rights in the invention.

### FIELD

This disclosure relates to methods of diagnosing a disease or condition associated with abnormal platelet activation in a subject. Also disclosed herein are methods for assessing the propensity of donor platelets to release an uptaken fluorescent false neurotransmitter (FFN).

### INTRODUCTION

Heparin induced thrombocytopenia (HIT) is a devastating immune-thrombotic disorder that affects 0.5-1% of patients exposed to the anticoagulant unfractionated heparin (heparin). This condition refers to a fall in blood platelet counts occurring in some patients who receive heparin therapy in any form. Two types of HIT exist, HIT-1 and HIT-2. HIT-1 is characterized by a brief and asymptomatic fall in the platelet count to as low as 100×10⁹/L. This condition resolves spontaneously on its own and does not require discontinuation of heparin. It is thought that this condition is caused by heparin-induced platelet clumping, no immune component of the disease has been identified, and complications of the condition are unusual. The second type of HIT is more deadly. HIT-2 has an immunologic component and is characterized by a profound fall in the platelet count (>50%) often after the fifth day of heparin therapy. In contrast to HIT-1, in which complications are rare, HIT-2 is usually accompanied by major arterial, venous or microvascular thrombosis, with loss of organ function or limb perfusion. Untreated, the condition can result in death. The incidence of the disease has varied widely, depending on the type of heparin, route of administration, and patient population.

HIT-2 is caused by antibodies that bind to platelet factor 4/heparin complexes (anti-PF4/heparin) resulting in activation of platelets and a hypercoagulable state. Formation of the PF4/heparin complex occurs optimally at equivalent stoichiometric concentrations of PF4 and heparin. When administered as a therapeutic anticoagulant, heparin levels range from 0.2 to 0.4 U/ml, or 100-200 nmol/L enabling PF4/heparin complex formation at optimal stoichiometric ratios that promote antibody formation. In patients such as those undergoing cardiac surgery, in vivo activation of platelets occurs, releasing PF4 into the circulation and increasing circulating PF4 levels (to about 75-100 nmol/L). However, due to high heparin levels used in cardiac surgery (2-5 U/mL or 1,000-2,500 nmol/L), PF4/heparin complexes do not form during cardiac surgery. Therefore, low doses of heparin (0-1 U/mL), but not high doses of heparin (2.5-100 U/mL), result in PF4/heparin complex formation and abnormal platelet activation. When heparin binds to PF4, it produces a conformational change in the protein, exposing antigenic epitopes to which anti-PF4/heparin antibodies bind. Diagnosis of HIT relies on the presence of certain clinical features and laboratory detection of anti-PF4/heparin antibodies showing heparin-dependent reactivity.

Laboratory testing of anti-PF4/heparin antibodies is performed using immunoassays or platelet activation assays. Immunoassays are the first line of testing and have the advantages of fast turn-around times (2-3 hours) due to automation, commercially availability, and very high sensitivity (>98%). The challenge with these diagnostic assays is that they also identify asymptomatic or (non-disease causing) anti-PF4/heparin antibodies that frequently occur after heparin exposure. Indeed, about 8-17% of patients exposed to heparin for medical treatment of venous thromboembolism or cardiac catheterization and about 30-50% of patients undergoing cardiac surgery develop anti-PF4/heparin antibodies that do not cause disease. Because of these high rates of seropositivity with drug exposure, a positive immunoassay cannot sufficiently distinguish individuals with and without HIT-2.

To overcome the poor specificity of anti-PF4/heparin antibodies, clinicians rely on platelet activation or functional assays for diagnostic confirmation. Unlike immunoassays, functional assays rely on cellular activation by anti-PF4/heparin antibodies. Functional assays use endpoints of platelet activation that occurs when anti-PF4/heparin antibodies are incubated with low dose heparin, but not high dose heparin. The endpoint of platelet activation varies by assay. Functional assays include release of radioactivity (¹⁴C-serotonin), flow cytometric detection of P-selectin, and light transmission. The functional assays have high sensitivity (˜90%) and high specificity (>95%) for disease causing antibodies.

The gold standard for platelet functional assays in HIT is the radiolabeled ¹⁴C-serotonin release assay (SRA). The SRA is performed by loading platelets with a radiolabeled (¹⁴C) serotonin for 30 minutes, washing unbound radiolabel, and incubating patient plasma with and without heparin at low or high concentrations. The assay endpoint is release of ¹⁴C in the supernatant of activated platelets. Because of its reliance on radioactivity and regulatory challenges of using radioactivity in clinical laboratories, this test is performed only in large commercial clinical laboratories and has a slow turnaround time (e.g., days).

Thus, there is a need for a non-radioactive functional assay for determining whether a patient has anti-heparin/PF4 antibodies that have platelet-activating properties.

### SUMMARY

In an aspect, the disclosure relates to a method for diagnosing a disease or condition associated with abnormal platelet activation in a subject, the method comprising: (a) providing a first solution comprising donor platelets; (b) forming a second solution by mixing at least some of the first solution with a fluorescent false neurotransmitter (FFN) and incubating for a period of time for the donor platelets to uptake the FFN, wherein the FFN is a compound of Formula I:

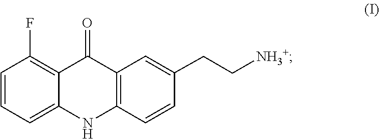

(c) forming a third solution by mixing some of the second solution with a biological sample from a subject and heparin, and incubating for a period of time, whereupon some of the donor platelets with uptaken FFN will release the FFN; (d) forming a fourth solution by mixing some of the second solution with a detergent, thereby lysing the donor platelets; (e) optionally forming a fifth solution by mixing some of the second solution with heparin; (f) centrifuging the third and fourth solutions, and at least one of the first, second and fifth solutions, and collecting the supernatant from each of the centrifuged solutions; (g) analyzing the collected supernatants by fluorometer to determine the fluorescence intensity of each collected supernatant; (h) calculating the percent release of the FFN from the donor platelets by using the following formula:

$$Percent Release = ( \frac{( Experimental MFI - Baseline MFI )}{( Maximum Signal MFI - Baseline MFI )} ) \times 100 ,$$

wherein the experimental MFI is the fluorescence intensity of the supernatant collected from the third solution, the maximum signal MFI is the fluorescence intensity of the supernatant collected from the fourth solution, and the baseline MFI either is the fluorescence intensity of the supernatant collected from the first solution, the second solution, or the fifth solution, or is the mean fluorescent intensity of the supernatants collected from more than one of the first solution, the second solution, and the fifth solution; wherein a percent release of at least 20% is indicative of abnormally activated platelets.

In some embodiments, the biological sample is obtained from a heparin-treated subject. In some embodiments, heparin treatment of the subject is ceased when the percent release of the FFN is at least 20%. In some embodiments, the biological sample is obtained from a subject suspected of having heparin-induced thrombocytopenia. In some embodiments, the disease or condition is heparin-induced thrombocytopenia. In some embodiments, the biological sample is a serum sample, a plasma sample, or isolated antibodies from a subject suspected of having heparin-induced thrombocytopenia. In some embodiments, the donor platelets are obtained from at least one healthy subject. In some embodiments, the donor platelets are washed and partially purified before providing the first solution. In some embodiments, the second solution is incubated at from about 18° C. to about 37° C. In some embodiments, the second solution is incubated for at least 15 minutes. In some embodiments, the third solution is incubated at from about 18° C. to about 37° C. In some embodiments, the third solution is incubated for at least 15 minutes. In some embodiments, the heparin is in an amount of from about 0.001 U/mL to about 2 U/mL. In some embodiments, the heparin is in an amount of from about 2.5 U/mL to about 100 U/mL. In some embodiments, forming the second solution further comprises incubating the donor platelets with a serotonin transporter inhibitor after the period of time to prevent further uptake of the FFN by the donor platelets. In some embodiments, the serotonin transporter inhibitor comprises imipramine, paroxetine, sertraline, fluvoxamine, desipramine, amitriptyline, zimelidine, clomipramine, or alprazolam. In some embodiments, forming the second solution further comprises washing the donor platelets after the period of time to remove any FFN not uptaken by the donor platelets.

In a further aspect, the disclosure relates to a method for assessing the propensity of donor platelets to release an uptaken fluorescent false neurotransmitter (FFN), the method comprising: (a) providing a first solution comprising donor platelets; (b) forming a second solution by mixing at least some of the first solution with the FFN and incubating for a period of time for the donor platelets to uptake the FFN, wherein the FFN is a compound of Formula I:

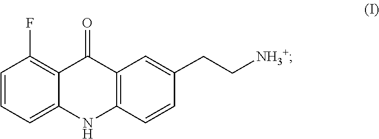

(c) forming a third solution by mixing some of the second solution with a biological sample from a subject and heparin, and incubating for a period of time, whereupon some of the donor platelets with uptaken FFN will release the FFN; (d) forming a fourth solution by mixing some of the second solution with a detergent, thereby lysing the donor platelets; (e) optionally forming a fifth solution by mixing some of the second solution with heparin; (f) centrifuging the third and fourth solutions, and at least one of the first, second and fifth solutions, and collecting the supernatant from each of the centrifuged solutions; (g) analyzing the collected supernatants by fluorometer to determine the fluorescence intensity of each collected supernatant.

In some embodiments, the biological sample is a serum sample, a plasma sample, or isolated antibodies from a subject suspected of having heparin-induced thrombocytopenia. In some embodiments, the biological sample is obtained from a heparin-treated subject. In some embodiments, the biological sample is obtained from a subject suspected of having heparin-induced thrombocytopenia. In some embodiments, the donor platelets are obtained from at least one healthy subject. In some embodiments, the donor platelets are washed and partially purified before providing the first solution. In some embodiments, the second solution is incubated at from about 18° C. to about 37° C. In some embodiments, the second solution is incubated for at least 15 minutes. In some embodiments, the third solution is incubated at from about 18° C. to about 37° C. In some embodiments, the third solution is incubated for at least 15 minutes. In some embodiments, the heparin is in an amount of from about 0.001 U/mL to about 2 U/mL. In some embodiments, the heparin is in an amount of from about 2.5 U/mL to about 100 U/mL. In some embodiments, forming the second solution further comprises incubating the donor platelets with a serotonin transporter inhibitor after the period of time to prevent further uptake of the FFN by the donor platelets. In some embodiments, the serotonin transporter inhibitor comprises imipramine, paroxetine, sertraline, fluvoxamine, desipramine, amitriptyline, zimelidine, clomipramine, or alprazolam. In some embodiments, forming the second solution further comprises washing the donor platelets after the period of time to remove any FFN not uptaken by the donor platelets.

The disclosure provides for other aspects and embodiments that will be apparent considering the following detailed description and accompanying figures.

### BRIEF DESCRIPTION OF THE DRAWINGS

FIG. 1 is a bar chart showing a dose dependent release of FFN246 from activated platelets. Platelets were incubated with varying concentrations of FFN246 as shown on the X-axis (black bars; 10 μM, 25 μM, 50 μM, or 100 μM) or with buffer (white bars). The platelets were washed and activated with A23187. Then, the fluorescence of the platelet releasate was measured to obtain the mean fluorescence intensity (MFI).

FIG. 2 is a bar chart showing the release of FFN246 from platelets exposed to a variety of agonists (Ca ionophore, ADP, ristocetin, collagen, IIa, Par4, and SFLLRN). Platelets were incubated with one of the agonists (x-axis) either with FFN246 (black bars) or buffer (white bars). Then, the fluorescence of the platelet releasate was measured to obtain the MFI.

FIG. 3 is a bar chart showing platelet activation by KKO, a HIT-like monoclonal antibody. Platelets were exposed to a variety of conditions (x-axis; Ca ionophore, collagen, SFLLRN, KKO, 0.5 U/mL heparin+KKO, 10 U/mL heparin+KKO, or 0.5 U/mL heparin+TRA, an isotype control antibody). Platelets were either incubated with FFN246 (black bars) or buffer (white bars). Then, the fluorescence of the platelet releasate was measured to obtain the MFI.

FIG. 4 is a bar chart showing the release of FFN246, but not FFN102, from platelets exposed to a Ca ionophore. Platelets were incubated with FFN246+Ca ionophore, FFN246+buffer, FFN102+Ca ionophore, FFN102+buffer, or buffer alone (x-axis). Then, the fluorescence of the platelet releasate was measured to obtain the MFI.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Abnormal platelet activation” or “pathological platelet activation” as used herein refers to platelets that are activated under conditions in which platelets in a healthy subject would not be activated. Abnormal platelet activation can induce occlusive thrombosis, resulting in ischemic events such as heart attack and stroke, and ultimately death.

As used herein, the term “biomarker” is any biomolecule that may provide biological information about the physiological state of a subject. In certain embodiments, the presence or absence of the biomarker may be informative. In other embodiments, the level of the biomarker may be informative. A biomarker may be a fluorescent false neurotransmitter (“FFN”), such as FFN246.

The terms “control,” “reference level,” and “reference” are used herein interchangeably. The reference level may be a predetermined value or range, which is employed as a benchmark against which to assess the measured result. “Control group” as used herein refers to a group of control subjects. The healthy or normal levels or ranges for a target or for a protein activity may be defined in accordance with standard practice. A control may be a subject or cell without a treatment or disease as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

As used herein, the term “heparin treatment” refers to a treatment regimen that includes administration of a heparin drug to a subject (e.g., a human subject). The term “heparin drug” refers to various heparins and heparin derivatives as known to those of skill in the art, including, but not limited to, heparin, unfractionated heparin, and low molecular weight heparins, including enoxaparin, dalteparin, and tinzaparin. Such drugs can be used for the treatment of various conditions, including conditions requiring anticoagulants (e.g., atrial fibrillation, pulmonary embolism, deep vein thrombosis, venous thromboembolism, congestive heart failure, stroke, myocardial infarction, and genetic or acquired hypercoagulability).

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Sample,” “test sample,” or “biological sample” as used herein can mean any sample in which the presence and/or level of a target is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

As used herein, the term “separate” or “purify” or the like are not used necessarily to refer to the removal of all materials other than the component of interest from a sample matrix. Instead, in some embodiments, the terms are used to refer to a procedure that enriches the amount of one or more components of interest relative to one or more other components present in the sample matrix. In some embodiments, a “separation” or “purification” may be used to remove or decrease the amount of one or more components from a sample that could interfere with the detection of the component of interest.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific antibody. The subject may be undergoing other forms of treatment.

“Treatment” or “treating” or “treatment” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Preventing the disease involves administering or avoiding administering heparin to a subject prior to onset of the disease. Suppressing the disease involves administering or stopping administration of heparin to a subject after induction of the disease but before its clinical appearance. Repressing or ameliorating the disease involves administering or stopping administration of heparin to a subject after clinical appearance of the disease.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, hematology, microbiology, genetics, and medicine described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Abnormal Platelet Activation

Resting platelets (non-activated platelets) express various cell surface receptors that respond to ligands generated or released during inflammation and damage to the vascular endothelium. Binding of the cell surface receptors results in activation of platelets. Activated platelets promote clot formation (thrombosis), broaden inflammatory responses, and contribute to wound healing. Impaired platelet activation can lead to uncontrolled bleeding and pathological or abnormal activation of platelets can cause several diseases or disorders (e.g., heparin induced thrombocytopenia (HIT), atherosclerosis, stroke, hypertension, and deep vein thrombosis) and is a common target of anti-thrombotic treatment for some of those diseases.

a. Heparin Induced Thrombocytopenia (HIT)

As known to those of skill in the art, sera from patients with HIT initiates platelet aggregation and secretion at therapeutic concentrations of heparin, but not at high concentrations of heparin. This assay has an advantage over other methods in that the assay does not use radiolabeled molecules. The method can be useful for other conditions and diseases associated with abnormal donor platelet activation.

### 3. Fluorescent False Neurotransmitters (FFN)

Serotonin, or 5-hydroxytryptamine (5-HT), is a neurotransmitter that is found in both the central and peripheral nervous systems. As a neurotransmitter, it subserves a variety of physiologic functions in the brain, among them, mood, cognition and perception, while in the peripheral nervous system, it effects smooth muscle function and platelet aggregation. Serotonin is taken up by cells through plasma membrane via a class of serotonin transporters (SERT) and binds to receptors belonging to the 5-HT receptor family (currently about 14 receptor subtypes are described). SERT is expressed on platelets and is primarily responsible for serotonin uptake.

Disclosed herein is an FFN that is a substrate for both SERT and the vesicular monoamine transporter 2 (VMAT2). The FFN may be used to diagnose a disease or condition associated with abnormal platelet activation in a subject. The FFN may include a compound of Formula I:

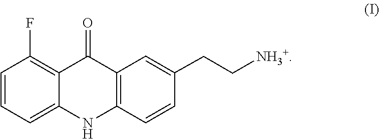

The compound of Formula I is described in Henke et al. ACS Chem Neurosci. 2018; 9(5): 925-934, which is incorporated herein by reference. The compound of Formula I is also referred to as “FFN246.” The compound of Formula I differs from serotonin in that Formula I is an acridone, is extremely fluorescent, and does not possess the pharmacological properties that serotonin has (e.g., no activity at 5-HT receptors and does not bind to neurotransmitter receptors). In addition, serotonin is an indole and is only mildly fluorescent.

The compound of Formula I was chemically designed as an optical substrate for the SERT. Chemically, it is an acridone functionalized with the ethylamine on the 2-position and fluorine atom on the 8-position of the C-ring. While the compound of Formula I shows SERT-dependent uptake as compared to other modified acridone compounds, it is also taken up to a lesser extent by dopamine transporters (DAT; 31% lower than SERT) and norepinephrine transporters (NET; 23% lower than SERT). In these studies, SERT-independent background uptake in brain tissue appears to be independent of the concentration of the compound of Formula I. The specificity of the compound of Formula I for the 5-HT receptor subclasses has not been studied. Even though the compound of Formula I was shown to be taken up by SERT into and retained by the cell bodies of neurons, the compound of Formula I is not taken up and retained in axons of neurons.

Another FFN described herein is the compound of Formula II:

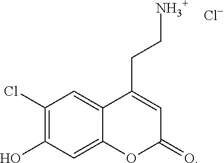 [SMILES: [NH3+]CCc1cc(=O)oc2cc(O)c(Cl)cc12 ; [Cl-]]

The compound of Formula II is described in Rodriguez et al., Proc Natl Acad Sci U S A. 2013; 110(3): 870-875, which is incorporated herein by reference. The compound of Formula II differs in structure from the compound of Formula I and is used in comparative experiments described below.

### 4. Methods

a. Methods of Diagnosing a Disease or Condition Associated with Abnormal Platelet Activation

Provided herein are methods for diagnosing a disease or condition associated with abnormal platelet activation in a subject. The methods may include method for diagnosing a disease or condition associated with abnormal platelet activation in a subject, the method comprising: (a) providing a first solution comprising donor platelets; (b) forming a second solution by mixing at least some of the first solution with the compound of Formula I and incubating for a period of time for the donor platelets to uptake the compound of Formula I; (c) forming a third solution by mixing some of the second solution with a biological sample from a subject and heparin, and incubating for a period of time, whereupon some of the donor platelets with uptaken compound of Formula I will release the compound of Formula I; (d) forming a fourth solution by mixing some of the second solution with a detergent, thereby lysing the donor platelets; (e) optionally forming a fifth solution by mixing some of the second solution with heparin; (f) centrifuging the third and fourth solutions, and at least one of the first, second and fifth solutions, and collecting the supernatant from each of the centrifuged solutions; (g) analyzing the collected supernatants by fluorometer to determine the fluorescence intensity of each collected supernatant; (h) calculating the percent release of the compound of Formula I from the donor platelets by using the following formula:

$$Percent Release = ( \frac{( Experimental MFI - Baseline MFI )}{( Maximum Signal MFI - Baseline MFI )} ) \times 100 ,$$

wherein the experimental MFI is the fluorescence intensity of the supernatant collected from the third solution, the maximum signal MFI is the fluorescence intensity of the supernatant collected from the fourth solution, and the baseline MFI either is the fluorescence intensity of the supernatant collected from the first solution, the second solution, or the fifth solution, or is the mean fluorescent intensity of the supernatants collected from more than one of the first solution, the second solution, and the fifth solution; wherein a percent release of at least 20% is indicative of abnormally activated platelets.

In some embodiments, the supernatant of the first solution is a first negative control; the supernatant of the second supernatant is a second negative control; and the supernatant of the fifth solution is a third negative control. The concentration of heparin in the fifth solution may be from about 2.5 U/mL to about 100 U/mL. In some embodiments, the supernatant of the fourth solution is a positive control.

In some embodiments, the biological sample may also include other components, such as solvents, buffers, anticlotting agents, and the like. In some embodiments, the biological sample can be one or more of whole blood, plasma, serum, urine, cerebrospinal fluid, tissue homogenate, saliva, amniotic fluid, bile, mucus, peritoneal fluid, or lymphatic fluid. In some embodiments, the biological sample is serum or plasma. In some embodiments, the biological sample may be a serum sample, a plasma sample, or isolated antibodies from a subject suspected of having heparin-induced thrombocytopenia. In some embodiments, the biological sample may be obtained from a drug-treated subject. For example, the biological sample can be obtained from a heparin-treated subject or a subject treated with a heparin analogue or derivative. In some embodiments, the biological sample may be obtained from a subject previously treated with heparin, unfractionated heparin, or low molecular weight heparins (LMWH), including enoxaparin, dalteparin, and tinzaparin. A subject may be treated with a high dose LMWH (2.5-100 U/mL) or low dose of LMWH (0.001-2 U/mL). The effects of high dose and low dose LMWH on abnormal platelet activation are similar to that of low dose and high dose heparin. In some embodiments, the biological sample may be obtained from a subject having or at risk for developing a disease or condition associated with abnormal platelet activation. For example, the biological sample can be obtained from a subject at risk for developing heparin-induced thrombocytopenia. A subject at risk for developing heparin-induced thrombocytopenia can include subjects previously or currently treated with heparin. The methods described herein are not limited to any particular volume of biological sample. In some embodiments, the biological sample may be at least about 1-100 μL, at least about 10-75 μL, or at least about 15-50 μL in volume. In certain embodiments, the biological sample may be at least about 20 μL in volume.

In some embodiments, the heparin treatment of the subject is ceased when the percent release of the compound of Formula I is at least 20%. In some embodiments, the disease or condition may be heparin-induced thrombocytopenia, atherosclerosis, stroke, hypertension, or deep vein thrombosis. In a particular embodiment, the disease or condition may be heparin-induced thrombocytopenia.

The donor platelets may be obtained from at least one healthy subject (e.g., a subject that does not have heparin-induced thrombocytopenia or a condition or disease associated with abnormal platelet activation). In some embodiments, the donor platelets may be pooled from any number of healthy subjects. For example, the donor platelets can be obtained from plasma from patients according to the method described below in Example 1. In some embodiments, the donor platelets are washed and/or purified before providing the first solution. In some embodiments, the amount of the compound of Formula I that can be used for forming the second solution can range from about 2.5 μM to about 100 μM, about 2.5 μM to about 50 μM, about 2.5 μM to about 25 μM, about 5 μM to about 100 μM, about 5 μM to about 50 μM, about 5 μM to about 25 μM, about 10 μM to about 100 μM, about 10 μM to about 50 μM, about 10 μM to about 25 μM, about 25 μM to about 100 μM, or about 25 μM to about 50 μM. In a particular embodiment, the amount of the compound of Formula I that can be used for forming the second solution can range from about 25 μM to about 100 μM. In some embodiments, incubating the second solution may be performed at from about 18° C. to about 37° C., about 20° C. to about 37° C., about 22° C. to about 37° C., about 24° C. to about 37° C., about 26° C. to about 37° C., about 28° C. to about 37° C., or about 30° C. to about 37° C. In some embodiments, incubating the second solution may be performed for at least 15 minutes, at least 20 minutes, at least 25 minutes, at least 30 minutes, at least 40 minutes, at least 45 minutes, at least 50 minutes, at least 55 minutes, or at least 60 minutes. In some embodiments, incubating the second solution may be performed for 15 minutes to 60 minutes.

The methods as described herein are not limited to any particular volume of donor platelets. Optionally, the donor platelets are provided in the form of a suspension in a buffer (e.g., an aqueous buffer). The buffer can include calcium ions (Ca²⁺) and at least one enzyme. In some embodiments, the enzyme is apyrase, a calcium-activated plasma membrane bound enzyme, which prevents adenosine diphosphate (ADP) accumulation. In some embodiments, the donor platelet suspension is at least about 25-250 μL, at least about 35-200 μL, at least about 45-150 μL, or at least about 50-100 μL in volume. In certain embodiments, the donor platelet suspension is at least about 75 μL in volume. The methods as described herein may comprise a concentration of donor platelets from about 150,000 platelets/μL to about 300,000 platelets/μL. Preferably, at least 150,000 platelets/μL may be used in a method as described herein.

In some embodiments, forming the second solution further comprises incubating the donor platelets with a serotonin transporter inhibitor to prevent further uptake of the compound of Formula I by the donor platelets. The serotonin transporter inhibitor may be any selective serotonin reuptake inhibitor (SSRI) such as imipramine, paroxetine, sertraline, fluvoxamine, desipramine, amitriptyline, zimelidine, clomipramine, or alprazolam. In some embodiments, forming the second solution further comprises washing the donor platelets after the period of time as described herein to remove any of the compound of Formula I that was not uptaken by the platelets.

In some embodiments, forming the first, second, third, fourth, and fifth solutions may include adding the solutions in separate wells of a culture dish. In some embodiments, incubating the third solution may be performed at from about 18° C. to about 37° C., about 20° C. to about 37° C., about 22° C. to about 37° C., about 24° C. to about 37° C., about 26° C. to about 37° C., about 28° C. to about 37° C., or about 30° C. to about 37° C. In some embodiments, incubating the third solution may be performed for at least 15 minutes, at least 20 minutes, at least 25 minutes, at least 30 minutes, at least 40 minutes, at least 45 minutes, at least 50 minutes, at least 55 minutes, or at least 60 minutes. In some embodiments, incubating the third solution may be performed for 15 minutes to 60 minutes.

In some embodiments, the heparin may be present in the third solution or in the fifth solution in an amount of from about 0.001 U/mL to about 100 U/mL, about 0.001 U/mL to about 50 U/mL, about 0.001 U/mL to about 25 U/mL, about 0.001 U/mL to about 10 U/mL, about 0.001 U/mL to about 5 U/mL, about 0.001 U/mL to about 2.5 U/mL, about 0.001 U/mL to about 1 U/mL, or about 2.5 U/mL to about 100 U/mL. In some embodiments, the heparin may be present in the third solution at a low concentration. A low concentration of heparin can include heparin provided in an amount of from about 0.001 U/mL to about 2 U/mL, from about 0.001 U/mL to about 1 U/mL, from about 0.005 U/mL to about 0.5 U/mL, or from about 0.01 U/mL to about 0.25 U/mL. In some embodiments, the heparin may be present in the third solution or fifth solution at a high concentration. A high concentration of heparin can include heparin provided in an amount of from about 2.5 U/mL to about 1000 U/mL, about 2.5 U/mL to about 100 U/mL, about 50 U/mL to about 1000 U/mL, about 75 U/mL to about 750 U/mL, or about 100 U/mL to about 500 U/mL. The methods as described herein are not limited to any particular volume of heparin. In some embodiments, the high concentration of heparin or the low concentration of heparin may be provided to the solutions as described herein in an amount of at least about 0.1-25 μL, at least about 0.5-20 μL, or at least about 1-15 μL in volume. In some embodiments, the third solution may comprise platelet factor 4 (PF4). The PF4 may be at a concentration of from about 1-5 μg/mL, about 5-10 μg/mL, or about 10-30 μg/mL. In some embodiments, if the third solution comprises PF4, then including heparin in the third solution is optional. Typically, PF4 is not needed in the third solution because the donor platelets produce endogenous PF4. In some embodiments, a blocking antibody to the Fc gamma RIIa receptor on platelets may be used instead of the high concentration of heparin.

b. Methods of Assessing the Propensity of Donor Platelets to Release an Uptaken FFN

Provided herein are methods assessing the propensity of donor platelets to release an uptaken compound of Formula I, the method may include: (a) providing a first solution comprising donor platelets as described herein; (b) forming a second solution by mixing at least some of the first solution with the compound of Formula I and incubating for a period of time for the donor platelets to uptake the compound of Formula I as described herein; (c) forming a third solution by mixing some of the second solution with a biological sample from a subject and heparin, and incubating for a period of time, whereupon some of the donor platelets with uptaken compound of Formula I will release the compound of Formula I as described herein; (d) forming a fourth solution by mixing some of the second solution with a detergent, thereby lysing the donor platelets as described herein; (e) optionally forming a fifth solution by mixing some of the second solution with heparin as described herein; (f) centrifuging the third and fourth solutions, and at least one of the first, second and fifth solutions, and collecting the supernatant from each of the centrifuged solutions as described herein; (g) analyzing the collected supernatants by fluorometer to determine the fluorescence intensity of each collected supernatant as described herein.

### 5. Kits for Diagnosing a Disease or Condition Associated with Abnormal Platelet Activation

Provided herein is a kit, which may be used to diagnose a disease or condition associated with abnormal platelet activation. The kit may comprise donor platelets, the compound of Formula I, and heparin for use in the methods as described above, and instructions for using said donor platelets, the compound of Formula I, and heparin. In some embodiments, the kit comprises a serotonin transporter inhibitor, and instructions for using the serotonin transporter inhibitor.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### 6. Examples

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

Reagents and Buffers. Acid-Citrate-Dextrose (500 mL): Na Citrate (trisodium citrate with 2·H₂O)—8.28 g, citric acid monohydrate—6.83 g, dextrose—9.39 g, ddH₂O-500 mL. Solution should be at pH 4.4. Pipes-Saline buffer (150 mM NaCl, 20 mM PIPES, pH 6.5), final volume of 100 mL: 0.6 g PIPES to 90 mL of H₂O, 3 mL of 5M NaCL, about 240 μL of 10N NaOH, and pH up to 6.5. Final volume was brought to 100 mL. 10× HEPES-Tyrodes buffer Stock Solution (500 mL): 17.85 g HEPES (150 mM), 2.28 g NaH₂PO₄ (33 mM), 40.3 g NaCl (1.38 M), 1.01 g KCl (27 mM), 9.9 g Dextrose (55 mM). 450 mL of H₂O was added and pH was brought to 7.4. The final volume was brought to 500 mL and it was stored at −20° C. 1 M Calcium Chloride (100 mL final volume): 14.7 g CaCl₂·2H₂O (Calcium Chloride dehydrate, Sigma C-5080) was dissolved in 100 mL H₂O. 0.1 M (100 mM) Magnesium Chloride (100 mL final volume): 2.033 g MgCl₂·6H₂O (Calcium Chloride dehydrate, Sigma C-5080) was dissolved in 100 mL H₂O. HEPES-Tyrodes buffer (1×: 15 mM HEPES, 3.3 mM NaH₂PO₄, 138 mM NaCl, 2.7 mM KCl, 5.5 mM Dextrose): 10 mL of 10×HEPES-Tyrodes stock solution, 90 mL of ddH₂O to total 100 mL of solution. The solution was brought to a pH of 7.4. HEPES-Tyrodes buffer (pH 7.4) with PGE1/apyrase (PGE1/Apy Buffer): apyrase (1 U/mL final concentration) and PGE1 (1 μM final concentration) were added to HEPES-Tyrodes buffer. For 20 mL HEPES-Tyrodes, 15 μL of 10 mM PGE1 was optionally added and 50 μL of 0.5 U/μL apyrase solution (final apyrase concentration should be 1.25 U/mL) was added. HEPES-Tyrodes buffer (pH 7.4) with Ca²⁺/Mg²⁺(Ca/Mg Buffer): For 30 mL HEPES-Tyrodes, 60 μL of 1M CaCl₂ and 30 μL of 1M MgCl₂ were added. FFN246 was a 10 mM stock solution with a molecular weight of 292.74 g/mol. FFN102 was a 10 mM stock solution (Sigma CAS 1234064-11-9) with a molecular weight of 353.68 g/mol.

Solutions. The following solutions were prepared: 100 mL of 1× HEPES-Tyrode's buffer, vortex, pH 7.4; HEPES-Tyrodes buffer (pH 7.4) with PGE1 (PGE1 Buffer); 30 mL of HEPES-Tyrode's buffer with Ca²⁺/Mg²⁺(Ca/Mg buffer); low dose heparin (LDH2, 1 U/mL) was 200 μL of 100 U/mL stock hep lock solution to 800 μL Hepes-Tyrodes; high dose heparin (HDH, 50 U/mL) from a stock of 1000 U/mL heparin; imipramine 1 mM stock solution; and FFN246 diluted to 10 mM solution in DMSO (F-5HT).

Drawing of donor blood and preparation of donor platelets. 10-20 cc of blood were drawn off into Acid-Citrate Dextrose. 20 mL of whole blood generated 8-10 mL of platelet rich plasma (PRP). The whole blood was spun down at 180 g for 20 minutes without using the brake. Donor PRP was separated and the needed PRP was calculated for each of the FFN246 and buffer conditions. 25 μM of FFN246 was added to the PRP condition labeled “FFN246”. 2.5 μL/mL of PGE1 was added to each PRP condition simultaneously. The mixture was incubated at 37° C. for 30 minutes in a H₂O bath. For example, for 2.5 mL PRP, 6.25 μL PGE1 and 6.25 μL FFN246 were added. FFN246 was not added to the buffer PRP condition.

The mixture was centrifuged at 700 g for 10 minutes with a brake at 22° C. to 26° C. The supernatant was discarded and the platelets were gently resuspended in PGE1/Apyrase buffer 1 mL per mL PRP for 15 minutes at room temperature. For example, 2.5 mL PRP was resuspended in 2.5 mL buffer. This was centrifuged at 700 g for 10 minutes at room temperature with a brake. The supernatant was discarded and FFN246 or platelets were resuspended into 2 mL/PRP of Ca/Mg buffer after using a pipette to completely remove PGE1/apyrase containing buffer above pellet. For example, 2.5 mL of platelets was resuspended with 5 mL of HT Ca/Mg.

When various conditions were tested, such as NP40, buffer alone, buffer+heparin, SFLLRN, and additional conditions dictated by an experiment, the following aliquots were added: agonist, plasma, or antibody; buffer added to agonist to make 50 μL total; and 150 μL of FFN246 buffer labeled platelets. The conditions were allowed to develop for 1 hour at 37° C. on an agitator. The conditions were then centrifuged at 700 g for 10 mins at room temperature with a brake. 75 μL of the supernatant was added per well in triplicate. Fluorometer analysis was ran within 1 hour at appropriate excitation/emission spectra (FFN246 ex/em (nm): 392/427).

Determination of mean baseline MFI. The mean baseline mean fluorescence intensity (MFI) was calculated by averaging the MFI values for three controls (n=3 for each control): (1) platelets loaded with FFN246 in buffer, (2) platelets loaded with FFN246 in buffer+100 U/mL heparin, and (3) platelets in buffer. The concentration in each control sample was substantially the same. Each control condition was incubated for 10 minutes with 1 mM of imipramine at 5 μL/mL to stop the uptake or release of FFN246 when present. Next, each control condition was centrifuged at 700 g for 10 minutes with a brake at room temperature. The supernatant was collected and 75 μL of the supernatant was added per well of a culture dish in triplicate. Fluorometer analysis was ran within 1 hour at appropriate excitation/emission spectra to determine the MFI for each control condition. The MFI for each control condition triplicate was averaged. Then, the average MFI for each control condition was averaged with the other control conditions to obtain the mean baseline MFI. The mean baseline MFI was set at 0% release of FFN246.

Determination of maximum signal. The maximum signal or the maximum uptake of FFN246 by the platelets was determined by lysing the platelets incubated with FFN246 in buffer that were centrifuged during determination of the mean baseline MFI. The centrifuged platelets or pelleted platelets were gently resuspended in PGE1/Apyrase buffer 1 mL per mL platelets for 15 minutes at room temperature. For example, 2.5 mL platelets were resuspended in 2.5 mL buffer. This was centrifuged at 700 g for 10 minutes at room temperature with a brake. The supernatant was discarded and the platelets were resuspended into 2 mL/platelets of Ca/Mg buffer after using a pipette to completely remove PGE1/apyrase containing buffer above the pellet. For example, 2.5 mL of platelets were resuspended with 5 mL of HT Ca/Mg. The platelets were lysed using NP40 lysis buffer to release the FFN246 that was taken up by the platelets into the buffer. The lysed platelet buffer mixture was centrifuged at 700 g for 10 minutes with a brake at room temperature. The supernatant was collected and 75 μL of the supernatant was added per well of a culture dish in triplicate. Fluorometer analysis was ran within 1 hour at appropriate excitation/emission spectra to determine the MFI for the maximum signal/uptake. The MFI for each triplicate was averaged to obtain the maximum signal MFI. The maximum signal MFI was set at 100% release of FFN246.

Determination of FFN246 percent release from a test sample. Using the MFI values from the mean baseline and the maximum signal, the percent release was calculated for various experimental conditions (e.g., HIT plasma+low or high heparin, healthy donor plasma+low or high heparin, etc.). The following formula was used:

$$Percent Release = ( \frac{( Experimental MFI - Mean Baseline MFI )}{( Maximum Signal MFI - Mean Baseline MFI )} ) \times 100$$

For example, if the maximum signal MFI is 300, the mean baseline MFI is 100, and the experimental MFI for a sample from a HIT patient incubated with 1 U/mL heparin is 250, then the percent release is 75%. For an experimental sample incubated with high dose heparin (e.g., 100 U/mL heparin), if the percent release is under 20% then the assay is valid and if the percent release is over 20% then the assay is indeterminate.

Use of NP40 as positive control. NP40 is a non-ionic, commercially available detergent used as a cell lysis buffer. Incubation with platelets results in cell membrane lysis and release of intracellular contents. Incubation with either NP40 or A23187 result in similar release of FFN246 from platelets. Due to A23187's intrinsic fluorescence at wavelengths similar to the excitation/emission spectrum of FFN246, NP40 has been favored as the positive control.

PF4 addition to assay conditions. Platelet factor 4 (PF4) is a 31.2 kDa protein synthesized primarily by megakaryocytes and stored in platelet alpha granules. Positively charged PF4 forms ultra large immune complexes with negatively charged heparin in patients who develop pathological heparin induced thrombocytopenia (HIT). Exogenous PF4 added to plasma conditions at 30 μg/ml in these experiments led to platelet activation in both healthy donor and HIT samples even in the absence of heparin addition.

### Example 2

### Determining Optimal FFN246 Concentrations

Calcium (Ca) ionophores mobilize calcium ions from intracellular storage sites or transport it through the plasma membrane to increase the concentration of cytoplasmic calcium ions (Ca²⁺) and thereby activate platelets. The Ca ionophore A23187 works by triggering release of Ca²⁺ from the dense tubular system of platelets and is a potent activator of platelets. A23187 was used to test if FFN246 loaded platelets would release FFN246 into the supernatant upon activation.

To demonstrate that FFN246 was taken up by platelets and released upon platelet activation by A23187, healthy donor platelets were incubated with and without FFN246 for 30 minutes, followed by addition of 1 mM imipramine for 10 minutes to block further reuptake of FFN246. Platelets were then washed twice in buffer containing prostaglandin E1 (PGE1) and apyrase to minimize platelet activation. The washed platelets were resuspended in Mg²⁺/Ca²⁺ buffer without inhibitors. Washed platelets were then incubated with A23187 for 45 minutes, after which platelets were centrifuged for 10 minutes at 576×g. Fluorescence in the platelet releasate/supernatant was measured in a BMG Labtech Fluostar Optima fluorometer at an appropriate emission/excitation spectra. Gain was set using the FFN246 releasate for each concentration at 50% of maximum signal.

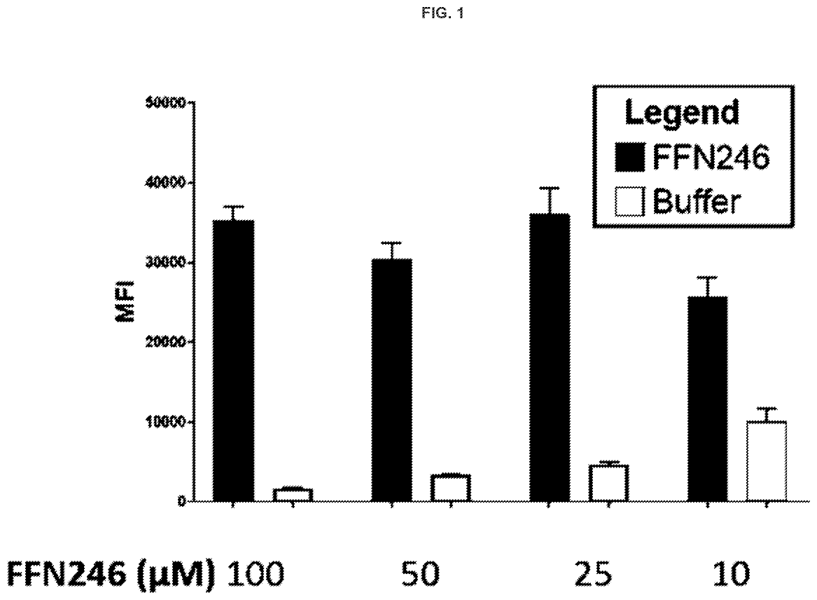

FIG. 1 shows that FFN246 at higher concentrations (25-100 μM) has an improved signal to background over A23187 alone or FFN246 at lower concentrations (10 μM). The MFI for FFN246+A23187 vs. buffer+A23187 at 100 μM was 35,405 vs. 1,676, respectively; at 50 μM was 30,470 vs. 3,461, respectively; at 25 μM was 36,124 v. 4,707, respectively; and, at 10 uM was 25,790 vs. 10,100, respectively.

These studies show that platelets incubated with FFN246 take up the fluorophore and secrete FFN246 upon platelet activation in a dose dependent manner.

### Example 3

### Release of FFN246 with Platelet Activation by Several Platelet Agonists

To determine if FFN246 loaded platelets release FFN246 with other platelet agonists and receptor mechanisms, FFN246 release was examined by incubating platelets with several platelet secretogogues, including collagen, IIa, PAR4, SFLLRN. Ristocetin, a negative control, causes platelet agglutination but does not cause platelet release. Collagen stimulates the GP VI receptor; ADP stimulates the P2Y1 and P2Y12 receptors; IIa activates PAR1 and PAR4 receptors; SFLLRN binds to the PAR1 receptor; PAR4 agonist binds the PAR4 receptor.

To examine the effect of various platelet agonists on FFN246 release, healthy donor platelets were washed and incubated with and without FFN246 (50 μM) for 30 minutes, followed by the addition of 1 mM imipramine for 10 minutes to block further reuptake. Platelets were then incubated with the various conditions: (1) A23187 10 μM; (2) buffer alone; (3) ADP 25 μM; (4) ristocetin 73 μM; (5) collagen 63 nM; (6) IIa 278 nM; (7) PAR4 peptide 200 μM; (8) SFLLRN 100 μM: and (9) buffer only platelets+A23187 (10 μM) for 45 minutes. Following incubation with agonists, platelets were centrifuged for 10 minutes at 576×g. Fluorescence in the platelet releasate (i.e. supernatant) was measured in a BMG Labtech Fluostar Optima fluorometer at an appropriate emission/excitation spectra. Gain was set using the FFN246 releasate for each concentration at 50% of maximum signal.

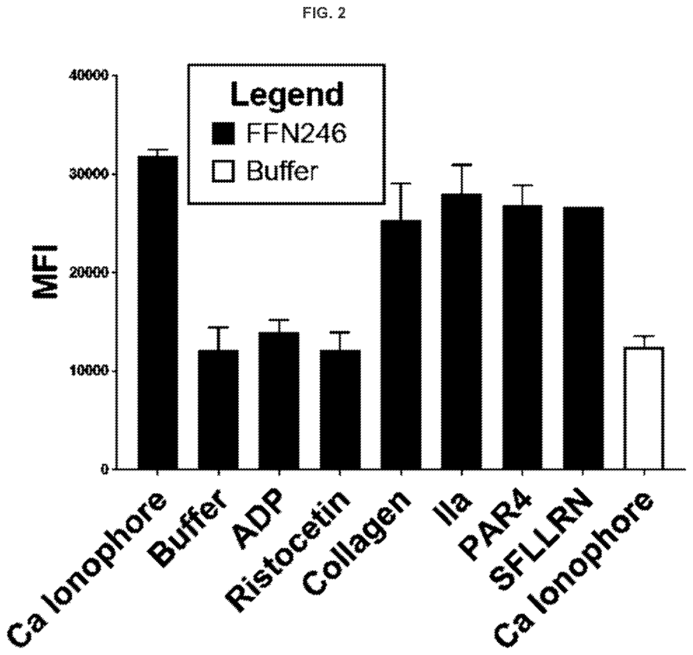

FIG. 2 shows the MFI of platelets exposed to a variety of agonists. Collagen, IIa, Par4, and SFLLRN demonstrate similar FFN246 release from exposed platelets. ADP demonstrated FFN246 release minimally higher than that of the buffer control. MFI for FFN246+calcium ionophore was 31924. MFI for FFN246+buffer was 12199. MFI for FFN246+ADP was 14013. MFI for FFN 246+ristocetin was 12247. MFI for FFN246+collagen was 24369. MFI for FFN246+IIa was 28080. MFI for FFN 246+Par4 was 26894. MFI for FFN246+SFLLRN was 26703. MFI for buffer+calcium ionophore was 12526.

These studies demonstrate that platelets are activated by platelet agonists thrombin or peptide derivatives and collagen. However, there was minimal fluorescent release with ADP. In studies not shown, we demonstrate that the decreased fluorescence with ADP is likely due to quenching effects of ADP.

### Example 4

### Platelet Activation with a HIT-Like Monoclonal Antibody

KKO, a HIT-like monoclonal antibody, activates platelets in the serotonin release assay at low heparin, but not high heparin doses.

To examine the effect of various platelet agonists on FFN246 release, healthy donor washed platelets were incubated with 50 μM FFN246 for 30 minutes, followed by addition of 1 mM imipramine for 10 minutes to block further reuptake. Platelets were then incubated with (1) A23187 10 μM; (2) collagen 63 nM; (3) SFLLRN 100 μM; (4) buffer; (5) KKO alone 25 μg/mL; (6) 0.5 U/mL heparin, 25 μg/ml KKO; (7) 10 U/mL heparin, 25 μg/mL KKO; (8) 0.5 U/mL heparin, 25 μg/mL TRA; and (9) A23187 10 μM (no FFN246). Incubation occurred for 45 minutes and platelets were centrifuged for 10 minutes at 576×g. Fluorescence in the platelet releasate was measured in a BMG Labtech Fluostar optima fluorometer at an appropriate emission/excitation spectra. Gain was set using the FFN246 releasate for each concentration at 50% of maximum signal.

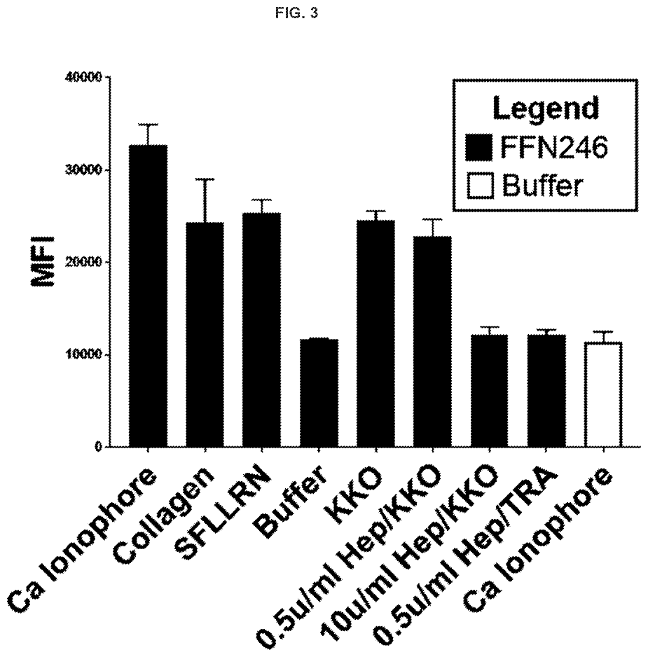

FIG. 3 shows the MFI of platelets exposed to the conditions. 0.5 U/mL of Heparin/KKO cause a large amount of FFN246 release, comparable to the platelet agonists, when compared to 10 U/mL heparin/KKO which had an MFI approaching that of the buffer conditions. MFI for FFN246+calcium ionophore was 32674. MFI for FFN246+collagen was 24388. MFI for FFN246+SFLLRN was 25426. MFI for FFN246+buffer was 11688. MFI for FFN246+KKO was 24604. MFI for FFN246+0.5 U/mL heparin and 25 μg/mL KKO was 22857. MFI for FFN246+10 U/mL heparin and KKO was 12184. MFI for FFN246+0.5 U/mL heparin and TRA was 12223. MFI for buffer+calcium ionophore was 11399.

These studies demonstrate that FFN246 is released in a heparin-dependent manner using a HIT like monoclonal antibody, but not an isotype. These findings suggest that the FFRA can be further developed as a diagnostic assay for HIT antibodies.

### Example 5

### Platelets Release FFN246, but Not FFN102

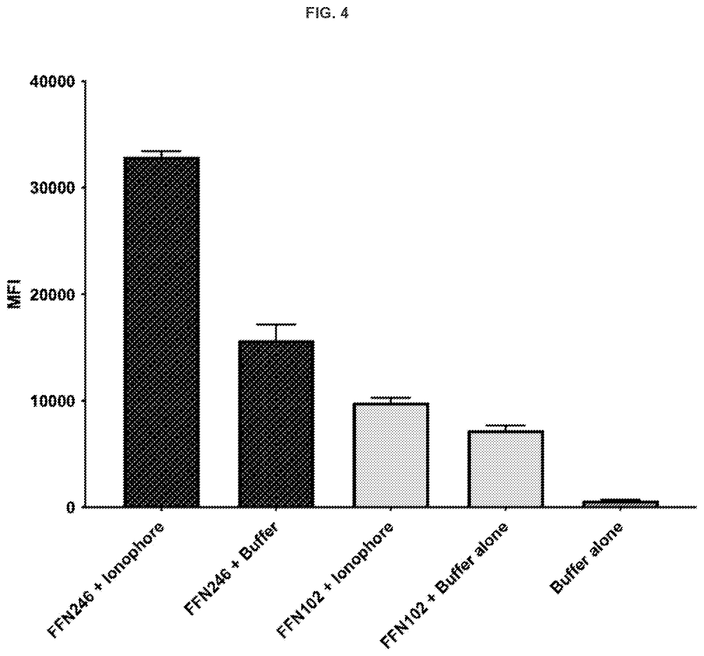

The amount of FFN246 released from platelets was compared to that of FFN102, a dopaminergic fluorophore. Healthy donor platelet rich plasma was incubated with 100 μM of FFN246, FFN102, or buffer alone. Incubation and washing occurred per the protocol described in Example 1. Resuspended platelets were added either to calcium ionophore or buffer conditions for both FFN246 and FFN102. Supernatant was collected following centrifugation and analyzed using fluorometry. Results demonstrated significant differences in MFI between FFN246 incubated with ionophore and with buffer (MFI 32901+448 v. 15,692+1203; FIG. 4). There were only small differences noted between the FFN102 platelets incubated with ionophore or buffer (MFI 9845+352 v. 7247+352; FIG. 4).

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A method for diagnosing a disease or condition associated with abnormal platelet activation in a subject, the method comprising:
(a) providing a first solution comprising donor platelets;
(b) forming a second solution by mixing at least some of the first solution with a fluorescent false neurotransmitter (FFN) and incubating for a period of time for the donor platelets to uptake the FFN, wherein the FFN is a compound of Formula I:

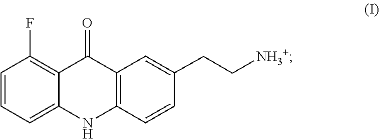

(c) forming a third solution by mixing some of the second solution with a biological sample from a subject and heparin, and incubating for a period of time, whereupon some of the donor platelets with uptaken FFN will release the FFN;
(d) forming a fourth solution by mixing some of the second solution with a detergent, thereby lysing the donor platelets;
(e) optionally forming a fifth solution by mixing some of the second solution with heparin;
(f) centrifuging the third and fourth solutions, and at least one of the first, second and fifth solutions, and collecting the supernatant from each of the centrifuged solutions;
(g) analyzing the collected supernatants by fluorometer to determine the fluorescence intensity of each collected supernatant;
(h) calculating the percent release of the FFN from the donor platelets by using the following formula:

$$Percent Release = ( \frac{( Experimental MFI - Baseline MFI )}{( Maximum Signal MFI - Baseline MFI )} ) \times 100 ,$$

wherein the experimental MFI is the fluorescence intensity of the supernatant collected from the third solution, the maximum signal MFI is the fluorescence intensity of the supernatant collected from the fourth solution, and the baseline MFI either is the fluorescence intensity of the supernatant collected from the first solution, the second solution, or the fifth solution, or is the mean fluorescent intensity of the supernatants collected from more than one of the first solution, the second solution, and the fifth solution;
wherein a percent release of at least 20% is indicative of abnormally activated platelets.

Clause 2. The method of clause 1, wherein the biological sample is obtained from a heparin-treated subject.

Clause 3. The method of clause 2, wherein heparin treatment of the subject is ceased when the percent release of the FFN is at least 20%.

Clause 4. The method of any one of clauses 1-3, wherein the biological sample is obtained from a subject suspected of having heparin-induced thrombocytopenia.

Clause 5. The method of any one of clauses 1-4, wherein the disease or condition is heparin-induced thrombocytopenia.

Clause 6. The method of any one of clauses 1-5, wherein the biological sample is a serum sample, a plasma sample, or isolated antibodies from a subject suspected of having heparin-induced thrombocytopenia.

Clause 7. The method of any one of clauses 1-6, wherein the donor platelets are obtained from at least one healthy subject.

Clause 8. The method of any one of clauses 1-7, wherein the donor platelets are washed and partially purified before providing the first solution.

Clause 9. The method of any one of clauses 1-8, wherein the second solution is incubated at from about 18° C. to about 37° C.

Clause 10. The method of any one of clauses 1-9, wherein the second solution is incubated for at least 15 minutes.

Clause 11. The method of any one of clauses 1-10, wherein the third solution is incubated at from about 18° C. to about 37° C.

Clause 12. The method of any one of clauses 1-11, wherein the third solution is incubated for at least 15 minutes.

Clause 13. The method of any one of clauses 1-12, wherein the heparin is in an amount of from about 0.001 U/mL to about 2 U/mL.

Clause 14. The method of any one of clauses 1-12, wherein the heparin is in an amount of from about 2.5 U/mL to about 100 U/mL.

Clause 15. The method of any one of clauses 1-14, wherein forming the second solution further comprises incubating the donor platelets with a serotonin transporter inhibitor after the period of time to prevent further uptake of the FFN by the donor platelets.

Clause 16. The method of clause 15, wherein the serotonin transporter inhibitor comprises imipramine, paroxetine, sertraline, fluvoxamine, desipramine, amitriptyline, zimelidine, clomipramine, or alprazolam.

Clause 17. The method of any one of clauses 1-16, wherein forming the second solution further comprises washing the donor platelets after the period of time to remove any FFN not uptaken by the donor platelets.

Clause 18. A method for assessing the propensity of donor platelets to release an uptaken fluorescent false neurotransmitter (FFN), the method comprising:
(a) providing a first solution comprising donor platelets;
(b) forming a second solution by mixing at least some of the first solution with the FFN and incubating for a period of time for the donor platelets to uptake the FFN, wherein the FFN is a compound of Formula I:

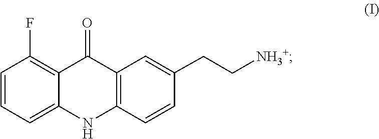

(c) forming a third solution by mixing some of the second solution with a biological sample from a subject and heparin, and incubating for a period of time, whereupon some of the donor platelets with uptaken FFN will release the FFN;
(d) forming a fourth solution by mixing some of the second solution with a detergent, thereby lysing the donor platelets;
(e) optionally forming a fifth solution by mixing some of the second solution with heparin;
(f) centrifuging the third and fourth solutions, and at least one of the first, second and fifth solutions, and collecting the supernatant from each of the centrifuged solutions;
(g) analyzing the collected supernatants by fluorometer to determine the fluorescence intensity of each collected supernatant.

Clause 19. The method of clause 18, wherein the biological sample is a serum sample, a plasma sample, or isolated antibodies from a subject suspected of having heparin-induced thrombocytopenia.

Clause 20. The method of clause 18 or clause 19, wherein the biological sample is obtained from a heparin-treated subject.

Clause 21. The method of any one of clauses 18-20, wherein the biological sample is obtained from a subject suspected of having heparin-induced thrombocytopenia.

Clause 22. The method of any one of clauses 18-21, wherein the donor platelets are obtained from at least one healthy subject.

Clause 23. The method of any one of clauses 18-22, wherein the donor platelets are washed and partially purified before providing the first solution.

Clause 24. The method of any one of clauses 18-23, wherein the second solution is incubated at from about 18° C. to about 37° C.

Clause 25. The method of any one of clauses 18-24, wherein the second solution is incubated for at least 15 minutes.

Clause 26. The method of any one of clauses 18-25, wherein the third solution is incubated at from about 18° C. to about 37° C.

Clause 27. The method of any one of clauses 18-26, wherein the third solution is incubated for at least 15 minutes.

Clause 28. The method of any one of clauses 18-27, wherein the heparin is in an amount of from about 0.001 U/mL to about 2 U/mL.

Clause 29. The method of any one of clauses 18-27, wherein the heparin is in an amount of from about 2.5 U/mL to about 100 U/mL.

Clause 30. The method of any one of clauses 18-29, wherein forming the second solution further comprises incubating the donor platelets with a serotonin transporter inhibitor after the period of time to prevent further uptake of the FFN by the donor platelets.

Clause 31. The method of clause 30, wherein the serotonin transporter inhibitor comprises imipramine, paroxetine, sertraline, fluvoxamine, desipramine, amitriptyline, zimelidine, clomipramine, or alprazolam.

Clause 32. The method of any one of clauses 18-31, wherein forming the second solution further comprises washing the donor platelets after the period of time to remove any FFN not uptaken by the donor platelets.

1. A method for diagnosing a disease or condition associated with abnormal platelet activation in a subject, the method comprising:
(a) providing a first solution comprising donor platelets;
(b) forming a second solution by mixing at least some of the first solution with a fluorescent false neurotransmitter (FFN) and incubating for a period of time for the donor platelets to uptake the FFN, wherein the FFN is a compound of Formula I: 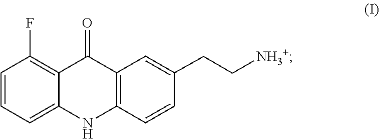
(c) forming a third solution by mixing some of the second solution with a biological sample from a subject and heparin, and incubating for a period of time, whereupon some of the donor platelets with uptaken FFN will release the FFN:
(d) forming a fourth solution by mixing some of the second solution with a detergent, thereby lysing the donor platelets;
(e) optionally forming a fifth solution by mixing some of the second solution with heparin;
(f) centrifuging the third and fourth solutions, and at least one of the first, second and fifth solutions, and collecting the supernatant from each of the centrifuged solutions;
(g) analyzing the collected supernatants by fluorometer to determine the fluorescence intensity of each collected supernatant;
(h) calculating the percent release of the FFN from the donor platelets by using the following formula:
$$Percent Release = ( \frac{( Experimental MFI - Baseline MFI )}{( Maximum Signal MFI - Baseline MFI )} ) \times 100 ,$$
wherein the experimental MFI is the fluorescence intensity of the supernatant collected from the third solution, the maximum signal MFI is the fluorescence intensity of the supernatant collected from the fourth solution, and the baseline MFI either is the fluorescence intensity of the supernatant collected from the first solution, the second solution, or the fifth solution, or is the mean fluorescent intensity of the supernatants collected from more than one of the first solution, the second solution, and the fifth solution;
wherein a percent release of at least 20% is indicative of abnormally activated platelets.

2. The method of claim 1, wherein the biological sample is obtained from a heparin-treated subject.

3. The method of claim 2, wherein heparin treatment of the subject is ceased when the percent release of the FFN is at least 20%.

4. The method of claim 1, wherein the biological sample is obtained from a subject suspected of having heparin-induced thrombocytopenia.

5. The method of claim 1, wherein the disease or condition is heparin-induced thrombocytopenia.

6. The method of claim 1, wherein the biological sample is a serum sample, a plasma sample, or isolated antibodies from a subject suspected of having heparin-induced thrombocytopenia.

7. The method of claim 1, wherein the donor platelets are obtained from at least one healthy subject.

8. The method of claim 1, wherein the donor platelets are washed and partially purified before providing the first solution.

9. The method of claim 1, wherein the second solution is incubated at from about 18° C. to about 37° C.

10. The method of claim 1, wherein the second solution is incubated for at least 15 minutes.

11. The method of claim 1, wherein the third solution is incubated at from about 18° C. to about 37° C.

12. The method of claim 1, wherein the third solution is incubated for at least 15 minutes.

13. The method of claim 1, wherein the heparin is in an amount of from about 0.001 U/mL to about 2 U/mL.

14. The method of claim 1, wherein the heparin is in an amount of from about 2.5 U/mL to about 100 U/mL.

15. The method of claim 1, wherein forming the second solution further comprises incubating the donor platelets with a serotonin transporter inhibitor after the period of time to prevent further uptake of the FFN by the donor platelets.

16. The method of claim 15, wherein the serotonin transporter inhibitor comprises imipramine, paroxetine, sertraline, fluvoxamine, desipramine, amitriptyline, zimelidine, clomipramine, or alprazolam.

17. The method of claim 1, wherein forming the second solution further comprises washing the donor platelets after the period of time to remove any FFN not uptaken by the donor platelets.

18. A method for assessing the propensity of donor platelets to release an uptaken fluorescent false neurotransmitter (FFN), the method comprising:
(a) providing a first solution comprising donor platelets;
(b) forming a second solution by mixing at least some of the first solution with the FFN and incubating for a period of time for the donor platelets to uptake the FFN, wherein the FFN is a compound of Formula I: 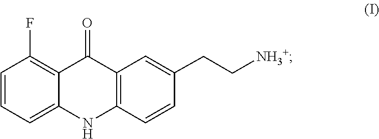
(c) forming a third solution by mixing some of the second solution with a biological sample from a subject and heparin, and incubating for a period of time, whereupon some of the donor platelets with uptaken FFN will release the FFN:
(d) forming a fourth solution by mixing some of the second solution with a detergent, thereby lysing the donor platelets;
(e) optionally forming a fifth solution by mixing some of the second solution with heparin;
(f) centrifuging the third and fourth solutions, and at least one of the first, second and fifth solutions, and collecting the supernatant from each of the centrifuged solutions;
(g) analyzing the collected supernatants by fluorometer to determine the fluorescence intensity of each collected supernatant.

19. The method of claim 18, wherein the biological sample is a serum sample, a plasma sample, or isolated antibodies from a subject suspected of having heparin-induced thrombocytopenia.

20. The method of claim 18, wherein the biological sample is obtained from a heparin-treated subject.

21. The method of claim 18, wherein the biological sample is obtained from a subject suspected of having heparin-induced thrombocytopenia.

22. The method of claim 18, wherein the donor platelets are obtained from at least one healthy subject.

23. The method of claim 18, wherein the donor platelets are washed and partially purified before providing the first solution.

24. The method of claim 18, wherein the second solution is incubated at from about 18° C. to about 37° C.

25. The method of claim 18, wherein the second solution is incubated for at least 15 minutes.

26. The method of claim 18, wherein the third solution is incubated at from about 18° C. to about 37° C.

27. The method of claim 18, wherein the third solution is incubated for at least 15 minutes.

28. The method of claim 18, wherein the heparin is in an amount of from about 0.001 U/mL to about 2 U/mL.

29. The method of claim 18, wherein the heparin is in an amount of from about 2.5 U/mL to about 100 U/mL.

30. The method of claim 18, wherein forming the second solution further comprises incubating the donor platelets with a serotonin transporter inhibitor after the period of time to prevent further uptake of the FFN by the donor platelets.

31. The method of claim 30, wherein the serotonin transporter inhibitor comprises imipramine, paroxetine, sertraline, fluvoxamine, desipramine, amitriptyline, zimelidine, clomipramine, or alprazolam.

32. The method of claim 18, wherein forming the second solution further comprises washing the donor platelets after the period of time to remove any FFN not uptaken by the donor platelets.
